(19) United States
(12) Patent Application Publication
(10) Pub. No.: US 2005/0222190 A1
(43) Pub. Date: Oct. 6, 2005

(54) 1,4-bis-N-oxide azaanthracenediones and the use thereof

(76) Inventors: John G. Curd, Hillsborough, CA (US); Robert L. Capizzi, Philadelphia, PA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/093,036

(22) Filed: Mar. 30, 2005

Related U.S. Application Data
(60) Provisional application No. 60/557,387, filed on Mar. 30, 2004.

(51) Int. Cl. A61K 31/473; C07D 221/22

(52) U.S. Cl. 514/290; 546/79

(57) ABSTRACT

Disclosed are compounds having Formula IV: 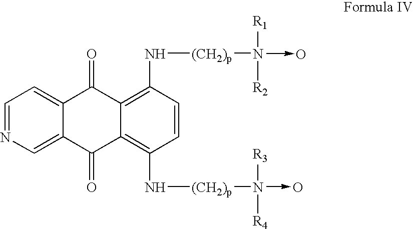
and pharmaceutically acceptable salts thereof, wherein R₁, R₂, R₃, R₄ and p are as defined herein, for use in methods for treating, preventing or ameliorating hyperproliferative disorders, such as cancer. The invention also relates to pharmaceutical compositions and formulations comprising a compound having Formula IV, and in combination with one or more other active agents and/or treatments.

### CROSS-REFERENCE TO RELATED APPLICATION

This application claims the benefit of U.S. Provisional Application No. 60/557,387, filed Mar. 30, 2004, the entirety of which is herein incorporated by reference.

### BACKGROUND OF THE INVENTION

1. Field of the Invention

The present invention relates to novel compounds having activity for treating hyperproliferative disorders, including neoplastic and non-neoplastic disorders, as well as certain inflammatory conditions. The invention also relates to pharmaceutical compositions and formulations comprising the novel compounds. Further, the invention relates to methods of using the novel compounds, alone or in combination with one or more other active agents or treatments, to treat hyperproliferative disorders, including various cancers.

2. Related Art

One in every four deaths in the United States is due to cancer, and is the second leading cause of death. U.S. Cancer Statistics Working Group; United States Cancer Statistics: 2000 Incidence, Atlanta (Ga.): Department of Health and Human Services, Centers for Disease Control and Prevention, and National Cancer Institute (2003). The National Cancer Institute reports that almost 10 million Americans have a history of invasive cancer, while the American Cancer Society estimates that in the year 2003, over 1.3 million Americans will receive a diagnosis of invasive cancer with over half of one million cases resulting in death. These statistics exclude the 1 million cases of basal and squamous cell skin cancers that are expected to diagnosed in the United States.

Cancers are classified based on the organ and cell tissue from which the cancer originates, including: (i) carcinomas (most common kind of cancer which originates in epithelial tissues, the layers of cells covering the body's surface or lining internal organs and various glands); (ii) leukemias (origination in the blood-forming tissues, including bone marrow, lymph nodes and the spleen); (iii) lymphomas (originates in the cells of the lymph system); (iv) melanomas (originates in the pigment cells located among the epithelial cells of the skin); and (v) sarcomas (originates in the connective tissues of the body, such as bones, muscles and blood vessels). (See Molecular Biology of the Cell: Third Edition, “Cancer,” Chapter 24, pp. 1255-1294, B. Alberts et al., (eds.), Garland Publishing, Inc., New York (1994); and Stedman's Pocket Medical Dictionary; Williams and Wilkins, Baltimore (1987)). Within these broad cancer classifications, there are over one hundred cancer subclassifications, such as breast, lung, pancreatic, colon, and prostate cancer, to name a few.

Cancer cells develop as a result of damage to the cell's DNA (i.e., altered DNA sequence or altered expression pattern) from exposure to various chemical agents, radiation, viruses, or when some not-yet-fully-understood internal, cellular signaling event occurs. Most of the time when a cell's DNA becomes damaged, the cell either dies or is able to repair the DNA. However, for cancer cells, the damaged DNA is not repaired and the cell continues to divide exhibiting modified cell physiology and function.

Neoplasms, or tumors, are masses of cells that result from an aberrant, accelerated rate of growth (i.e., hyperproliferative cell growth). As long as the tumor cells remain confined to a single mass, the tumor is considered to be benign. However, a cancerous tumor has the ability to invade other tissues and is termed malignant. In general, cancer cells are defined by two heritable properties: the cells and their progeny 1) reproduce in defiance of normal restraints, and 2) invade and colonize the territories of other cells.

Cancerous tumors are comprised of a highly complex vasculature and differentiated tissue. A large majority of cancerous tumors have hypoxic components, which are relatively resistant to standard anti-cancer treatment, including radiotherapy and chemotherapy. Brown, J. M. “The Hypoxic Cell: A Target for Selective Cancer Therapy—Eighteenth Bruce F. Cain Memorial Award Lecture” Cancer Research 59:5863-5870 (1999); and Kunz, M. and Ibrahim, S. M. “Molecular responses to hypoxia in tumor cells” Molecular Cancer 2:1-13 (2003). Thomlinson and Gray presented the first anatomical model of a human tumor that describes a 100 to 150 μm thick hypoxic layer of tissue located between the blood vessels and necrotic tumor tissues.

Research has shown that the hypoxic tissues within a number of cancerous tumors promote the progression of the cancer by an array of complex mechanisms. See, Brown, J. M., supra, and Kunz, M. and Ibrahim, S. M., supra. Among these are activation of certain signal transduction pathways and gene regulatory mechanisms, induction of selection processes for gene mutations, tumor cell apoptosis and tumor angiogenesis. Most of these mechanisms contribute to tumor progression. Therefore, tissue hypoxia has been regarded as a central factor for tumor aggressiveness and metastasis. Thus, therapies that target hypoxic tissues within a tumor would certainly provide improved treatments to patients suffering from tumor-related cancers and/or disorders.

In addition to cancer, there exists a number of hyperproliferative diseases and/or disorders that are associated with the onset of hypoxia in a given tissue. For example, Shweiki et al. explains that inadequate oxygen levels often lead to neovascularization in order to compensate for the needs of the hypoxic tissue. Neovascularization is mediated by expression of certain growth factors, such as vascular endothelial growth factor (VEGF). Shweiki, D. et al., “Vascular endothelial growth factor induced by hypoxia may mediate hypoxia-initiated angiogenesis” Nature 359:843-845 (1992). However, when certain tissues or growth factors are either directly or indirectly upregulated in response to hypoxia without sufficient feedback mechanisms for controlling tissue expression, various diseases and/or disorders may ensue (i.e., by hypoxia-aggravated hyperproliferation). By way of example, certain hypoxia-aggravated hyperproliferative diseases and/or disorders having over-expressed levels of VEGF include ocular angiogenic diseases, such as age-related macular degeneration and diabetic retinopathy, as well as cirrhosis of the liver. See Frank, R. N. “Growth Factors in Age-Related Macular Degeneration: Pathogenic and Therapeutic Implications” Ophthalmic Research 29:341-353 (1997); Ishibashi, T., et al. “Expression of vascular endothelial growth factor in experimental choroidal neovascularization” Graefe's Archive for Clinical and Experimental Ophthamology 235 (3): 159-167 (1997); Corpechot, C. et al. “Hypoxia-Induced VEGF and Collagen I Expressions Are Associated With Angiogenesis and Fibrogenesis in Experimental Cirrhosis,” Hepatology 35 (5):1010-1021 (2002).

PCT Published International Application WO 92/15300 describes 6,9-bis(substituted-amino)benzo[g]isoquinoline-5,10-diones having Formula I. 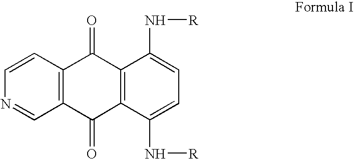
wherein R is C₁-C₁₀ alkyl; phenyl or C₇-C₁₀ aralkyl; C₂-C₁₀ alkyl substituted with one or two substituents selected from the group consisting of OR₁ and —NR₂R₃; C₂-C₁₀ alkyl interrupted by one or two oxygen atoms or by a member selected from the group consisting of —NR₄—, cis-CH═CH, trans-CH═CH— and —C≡C—, and optionally substituted with one or two hydroxy (OH) or —NR₂R₃ groups; and wherein
R₁ is selected from the group consisting of hydrogen, C₁-C₆ alkyl, phenyl, C₇-C₁₀ aralkyl, —CHO, —COR₅—, —COOR₅, —S(O₂)R₅ and C₂-C₆ alkyl optionally substituted with —NR₂R₃;
R₂ and R₃ are the same or different and are selected from the group consisting of hydrogen, C₁-C₁₀ alkyl, C₇-C₁₀ aralkyl, phenyl, C₂-C₁₀ alkyl substituted with one or two hydroxy (OH) groups, —CHO, —COR₅, —COOR₅, and —S(O₂)R₅, R₂ and R₃ taken together with the nitrogen atom to which they are bound form an ethyleneimine ring or a 5- or 6-membered aromatic or non-aromatic heterocyclic ring optionally containing another heteroatom selected from the group consisting of sulfur, oxygen and nitrogen, R₂ is H and R₃ is —C(═NH)NH₂ or R₂ is —C(═NH)NH₂ and R₃ is H;
   R₄ is selected from the group consisting of hydrogen, C₁-C₁₀ alkyl, C₂-C₁₀ hydroxyalkyl, C₂-C₁₀ alkyl substituted with —NR₂R₃, C₇-C₁₀ aralkyl, phenyl, —COR₅, —COOR₅ and —S(O₂)R₅;
   R₅ is selected from the group consisting of C₁-C₁₀ alkyl, C₇-C₁₀ aralkyl, alpha-, beta-, or gamma-naphthyl, phenyl, o-, m-, or p-tolyl as free bases and their salts with pharmaceutically acceptable acids. The compounds having Formula I are described as having cytostatic and anti-tumor activity.

PCT Published International Application WO 92/15566 describes nitrogen oxides of aza and diaza-anthracenediones having Formula II 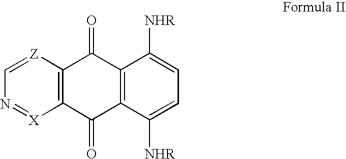
wherein X, Y and Z are CH, N or N→O thus forming an azine or diazine ring, with the proviso that:
at least one of X, Y and Z is N→O;
R is hydrogen, (C₁-C₁₀)-alkyl, phenyl, (C₇-C₁₀)aralkyl, (C₂-C₁₀)-alkyl having one or two substituents selected from —OR₁, —NR₂R₃; 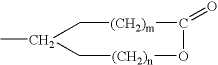
   (C₂-C₁₀)-alkyl chain optionally interrupted by one or two oxygen atoms or by one member selected from the group consisting of —NR₄—, cis-CH═CH—, trans-CH═CH—, —C≡C— and optionally substituted by one or two hydroxy or —NR₂R₃ groups;
R₁ is hydrogen, phenyl, (C₇-C₁₀)-aralkyl, (C₁-C₁₀)-alkyl, or a group of formula —C(O)H, —C(O)R₅, —C(O)OR₅, C₂-C₆ alkyl optionally substituted by —NR₂R₃;
R₂ and R₃, which can be the same or different, are hydrogen, (C₁-C₁₀)-alkyl, (C₂-C₁₀)-alkyl substituted with one or two hydroxy groups, (C₇-C₁₀)-aralkyl, phenyl, or one of R₂ or R₃ is a group of formula —C(O)H, —C(O)R₅, —C(O)OR₅, —C(O)NH—R₅, —SO₂—R₅; or R₂ and R₃, together with the nitrogen atom to which they are linked, form an aziridine ring, or a 5- or 6-membered aromatic or non aromatic heterocyclic ring which might contain another heteroatom such as nitrogen, oxygen or sulfur;
R₄ is hydrogen, (C₁-C₁₀)-alkyl, (C₂-C₁₀)-hydroxyalkyl, a C₂-C₁₀ alkyl substituted by —NR₂R₃, (C₁-C₇)-aralkyl, phenyl, —C(O)H, —C(O)R₅, —C(O)OR₅, —SO₂—R₅;
R₅ is (C₁-C₁₀)-alkyl; (C₇-C₁₀)-aralkyl; phenyl; m and n are independently zero or the integers 1 and 2, with the proviso that m and n cannot be zero at the same time; and the pharmaceutically acceptable salts thereof. The compounds having Formula II are described as having antitumor activity in vitro and in vivo.

U.S. Pat. No. 5,132,327 describes a group of anthraquinone prodrug compounds having Formula III: 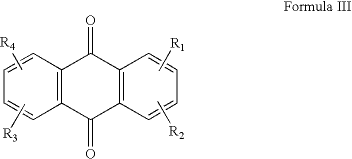
in which R₁, R₂, R₃ and R₄ are each separately selected from the group consisting of hydrogen, X, NH-A-NHR and NH-A-N(O)R′R″ wherein X is hydroxy, halogeno, amino, C₁₋₄ alkoxy or C₂₋₈ alkanoyloxy, A is a C₂₋₄ alkylene group with a chain length between NH and NHR or N(O)R′R″ of at least 2 carbon atoms and R, R′ and R″ are each separately selected from the group consisting of C₁₋₄ alkyl groups and C₂₋₄ hydroxyalkyl and C₂₋₄ dihydroxyalkyl groups in which the carbon atom attached to the nitrogen atom does not carry a hydroxy group and no carbon atom is substituted by two hydroxy groups, or R′ and R″ together are a C₂₋₆ alkylene group which with the nitrogen atom to which R′ and R″ are attached forms a heterocyclic group having 3 to 7 atoms in the ring, but with the proviso that at least one of R₁ to R₄ is a group NH-A-N(O)R′R″, the compound optionally being in the form of a physiologically acceptable salt. The compounds having Formula III are described as being useful in the treatment of cancer.

### BRIEF SUMMARY OF THE INVENTION

The present invention is related to compounds, compositions and methods for treating hyperproliferative disorders, such as cancer.

One aspect of the invention is drawn to compounds having Formula IV: 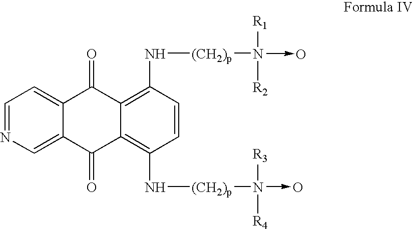
or a pharmaceutically acceptable salt thereof, wherein:
R₁, R₂, R₃ and R₄ are independently C₁₋₄ alkyl, C₂₋₄ hydroxyalkyl, C₂₋₄ dihydroxyalkyl in which the carbon atom attached to the nitrogen atom does not carry a hydroxy group and no carbon atom is substituted by two hydroxy groups,
or R₁ and R₂ can be taken together as a C₂₋₆ alkylene or C₄₋₆ alkylidene with the nitrogen atom to which R₁ and R₂ are attached to form a heterocycle having 3 to 7 atoms in the ring;
and/or R₃ and R₄ can be taken together as a C₂₋₆ alkylene or C₄₋₆ alkylidene with the nitrogen atom to which R₃ and R₄ are attached to form a heterocycle having 3 to 7 atoms in the ring; and
p is 2 to 4.

According to another aspect of the invention, a therapeutically effective amount of a compound having Formula IV is provided in the form of a pharmaceutical composition having at least one pharmaceutically acceptable diluent or binder.

Another aspect of the invention, methods for treating, preventing or ameliorating hyperproliferative disorders are provided, wherein a therapeutically effective amount of a compound having Formula IV, or a pharmaceutically acceptable salt thereof, is administered to an animal in need thereof. Additionally, the invention can be practiced by formulation of a compound having Formula IV and at least one or more other active agents optionally as part of a single pharmaceutical composition.

While not wishing to be bound in theory, it is believed that the N-oxide compounds of the invention are non-cytotoxic prodrugs. The N-oxide compounds are believed to be activated under hypoxic conditions within the target tissues (i.e., reduced at the nitrogen atom), followed by intercalation between the base pairs in the host cell DNA. It is contemplated the putative targets of the compounds for facilitating cell toxicity include, DNA reactivity, helicases, microtubules, protein kinase C, and topoisomerase II. Since a number of pathological tissues have significant hypoxic components which promote hyperproliferation, it is believed that this portion of tissue is preferentially targeted. It is also believed that compounds having Formula IV are useful for the treatment, prevention or amelioration of any number of hypoxia-aggrevated hyperproliferative diseases and/or disorders. Such diseases and/or disorders include, without limitation, age-related macular degeneration, various cancers, Crohn's disease, cirrhosis, chronic inflammatory-related disorders, diabetic retinopathy, granulomatosis, immune hyperproliferation associated with organ or tissue transplantation, inflammatory bowel disease, psoriasis, rheumatoid arthritis, systemic lupus erythematosus (SLE), vascular hyperproliferation secondary to retinal hypoxia, vascular hyperproliferation secondary to retinal hypoxia and vasculitis.

### DETAILED DESCRIPTION OF THE INVENTION

In some embodiments, the present invention is directed to compounds having Formula IV: 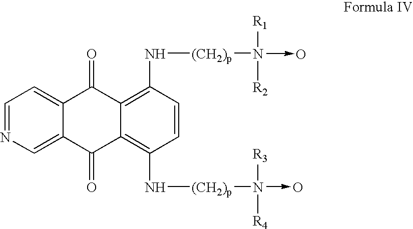
or a pharmaceutically acceptable salt thereof, wherein:
R₁, R₂, R₃ and R₄ are independently C₁₋₄ alkyl, C₂₋₄ hydroxyalkyl, C₂₋₄ dihydroxyalkyl in which the carbon atom attached to the nitrogen atom does not carry a hydroxy group and no carbon atom is substituted by two hydroxy groups,
or R₁ and R₂ can be taken together as a C₂₋₆ alkylene or C₄₋₆ alkylidene with the nitrogen atom to which R₁ and R₂ are attached to form a heterocycle having 3 to 7 atoms in the ring;
and/or R₃ and R₄ can be taken together as a C₂₋₆ alkylene or C₄₋₆ alkylidene with the nitrogen atom to which R₃ and R₄ are attached to form a heterocycle having 3 to 7 atoms in the ring; and
p is 2 to 4.

In certain embodiments, the invention is directed to compounds having Formula IV, wherein R₁, R₂, R₃ and R₄ are independently C₁₋₄ alkyl, C₂₋₄ hydroxyalkyl, C₂₋₄ dihydroxyalkyl in which the carbon atom attached to the nitrogen atom does not carry a hydroxy group and no carbon atom is substituted by two hydroxy groups.

Some embodiments of the invention are directed to compounds having Formula IV, wherein R₁ and R₂ are C₁₋₄ alkyl.

In some other embodiments, the invention is directed to compounds having Formula IV, wherein R₁ and R₂ are C₁₋₄ alkyl, and C₁₋₄ alkyl is selected from the group consisting of methyl, ethyl, propyl, isopropyl, and butyl. In certain instances of the invention, R₁ and R₂ will each be methyl.

Some embodiments of the invention is directed to compounds having Formula IV, wherein R₃ and R₄ are C₁₋₄ alkyl.

In some other embodiments, the invention is directed to compounds having Formula IV, wherein R₃ and R₄ are C₁₋₄ alkyl, and C₁₋₄ alkyl is selected from the group consisting of methyl, ethyl, propyl, isopropyl, and butyl. In certain instances of the invention, R₃ and R₄ will each be methyl.

In other embodiments, the invention is directed to compounds having Formula IV, wherein R₁ and R₂ are taken together as a C₂₋₆ alkylene or C₄₋₆ alkylidene with the nitrogen atom to which R₁ and R₂ are attached to form a heterocycle having 3 to 7 atoms in the ring.

In yet other embodiments, the invention is directed to compounds having Formula IV, wherein R₁ and R₂ are taken together with the nitrogen atom to which R₁ and R₂ are attached to form a hetercycle selected from the group consisting of piperidinyl, morpholinyl and aziridinyl.

In certain other embodiments, the invention is directed to compounds having Formula IV, wherein R₃ and R₄ are taken together as a C₂₋₆ alkylene or C₄₋₆ alkylidene with the nitrogen atom to which R₃ and R₄ are attached to form a heterocycle having 3 to 7 atoms in the ring.

In yet other embodiments, the invention is directed to compounds having Formula IV, wherein R₃ and R₄ are taken together with the nitrogen atom to which R₃ and R₄ are attached to form a hetercycle selected from the group consisting of piperidinyl, morpholinyl and aziridinyl.

According to another embodiment, the invention is directed to the following individual compounds:
1,4-bis-{[2-(dimethylamino-N-oxide)ethyl]amino}-6-azaanthracene-9,10-dione;
1,4-bis-{[2-(diethylamino-N-oxide)ethyl]amino}-6-azaanthracene-9,10-dione;
1,4-bis-{[2-(dipropylamino-N-oxide)ethyl]amino}-6-azaanthracene-9,10-dione;
1,4-bis-{[2-(diisopropylamino-N-oxide)ethyl]amino}-6-azaanthracene-9,10-dione;
1,4-bis-{[2-(dibutylamino-N-oxide)ethyl]amino}-6-azaanthracene-9,10-dione;
1,4-bis-{[2-(dipiperidin-1-yl-N-oxide)ethyl]amino}-6-azaanthracene-9,10-dione;
1,4-bis-{[2-(dimorpholin-4-yl-N-oxide)ethyl]amino}-6-azaanthracene-9,10-dione;
1,4-bis-{[2-(aziridin-1-yl-N-oxide)ethyl]amino}-6-azaanthracene-9,10-dione;
1,4-bis-{[3-(dimethylamino-N-oxide)propyl]amino}-6-azaanthracene-9,10-dione;
1,4-bis-{[3-(diethylamino-N-oxide)propyl]amino}-6-azaanthracene-9,10-dione;
1,4-bis-{[3-(dipropylamino-N-oxide)propyl]amino}-6-azaanthracene-9,10-dione;
1,4-bis-{[3-(dibutylamino-N-oxide)propyl]amino}-6-azaanthracene-9,10-dione;
1,4-bis-{[3-(diisopropylamino-N-oxide)propyl]amino}-6-azaanthracene-9,10-dione;
1,4-bis-{[3-(dipiperidin-1-yl-N-oxide)propyl]amino}-6-azaanthracene-9,10-dione;
1,4-bis-{[3-(dimorpholin-4-yl-N-oxide)propyl]amino}-6-azaanthracene-9,10-dione; and
1,4-bis-{[3-(aziridin-1-yl-N-oxide)propyl]amino}-6-azaanthracene-9,10-dione.

According to another aspect of the invention, a therapeutically effective amount of a compound having Formula IV, or a pharmaceutically acceptable salt thereof, is provided in the form of a pharmaceutical composition having at least one pharmaceutically acceptable diluent or binder.

In a further aspect of the invention, the pharmaceutical composition comprises a compound having Formula IV and at least one other active agent. In certain instances, the at least one other active agent is a chemotherapeutic agent or active vitamin D compound. Compounds having Formula IV may be formulated in a single formulation with the other active agent(s), or formulated independently.

The term “pharmaceutical composition” as used herein is to be understood as defining compositions of which the individual components or ingredients are themselves pharmaceutically acceptable, e.g., where oral administration is foreseen, acceptable for oral use; where topical administration is foreseen, topically acceptable; and where intravenous administration is foreseen, intravenously acceptable.

According to one aspect of the invention, methods for treating, ameliorating or preventing hyperproliferative disorders are provided, wherein a therapeutically effective amount of a compound having Formula IV, or a pharmaceutically acceptable salt thereof, is administered to an animal in need thereof. In certain aspects of the invention, the hyperproliferative disorder is cancer. In certain other aspects of the invention, the hyperproliferative disorder is any one of age-related macular degeneration, Crohn's disease, cirrhosis, chronic inflammatory-related disorders, diabetic retinopathy, granulomatosis, immune hyperproliferation associated with organ or tissue transplantation, an immunoproliferative disease or disorder, e.g., inflammatory bowel disease, psoriasis, rheumatoid arthritis, systemic lupus erythematosus (SLE), vascular hyperproliferation secondary to retinal hypoxia, or vasculitis.

A further aspect of the invention relates to methods for treating, ameliorating or preventing a hyperproliferative disorder comprising administering a therapeutically effective amount of a compound having Formula IV, or a pharmaceutically acceptable salt thereof, in combination with at least one other active agent or treatment to a patient in need thereof.

Hyperproliferative disorders which can be treated with the compounds having Formula IV include any hypoxia-aggravated hyperproliferative disease and/or disorder, such as any number of cancers. Generally, such cancers include without limitation cancers of the bladder, brain, breast, cervix, colon, endometrium, esophagus, head and neck, kidney, larynx, liver, lung, oral cavity, ovaries, pancreas, prostate, skin, stomach, and testis. Certain of these cancers may be more specifically referred to as acute and chronic lymphocytic leukemias, acute granulocytic leukemia, adrenal cortex carcinomas, bladder carcinomas, breast carcinomas, cervical carcinomas, cervical hyperplasia, choriocarcinomas, chronic granulocytic leukemia, chronic lymphocytic leukemia, colon carcinomas, endometrial carcinomas, esophageal carcinomas, essential thrombocytosis, genitourinary carcinomas, hairy cell leukemia, head and neck carcinomas Hodgkin's disease, Kaposi's sarcoma, lung carcinomas, lymphoma, malignant carcinoid carcinomas, malignant hypercalcemia, malignant melanomas, malignant pancreatic insulinoma, medullary thyroid carcinoma, melanoma, multiple myeloma, mycosis fingoides, myeloid and lymphocytic leukemia, neuroblastoma, non-Hodgkin's lymphomas, osteogenic sarcoma, ovarian carcinomas, pancreatic carcinomas, polycythemia vera, primary brain carcinomas, primary macroglobulinemia, prostatic carcinomas, renal cell carcinomas, rhabdomyosarcoma, skin cancer, small-cell lung carcinomas, soft-tissue sarcomas, squamous cell carcinoma, stomach carcinomas, testicular carcinomas, thyroid carcinomas, and Wilms' tumor.

Animals which may be treated according to the present invention include all animals which may benefit from administration compounds having Formula IV. Such animals include humans, pets such as dogs and cats, and veterinary animals such as cows, pigs, sheep, goats and the like.

Compounds having Formula IV can be prepared in part as described in PCT Published International Application WO 92/15300, and as illustrated by exemplary reactions in Scheme I:

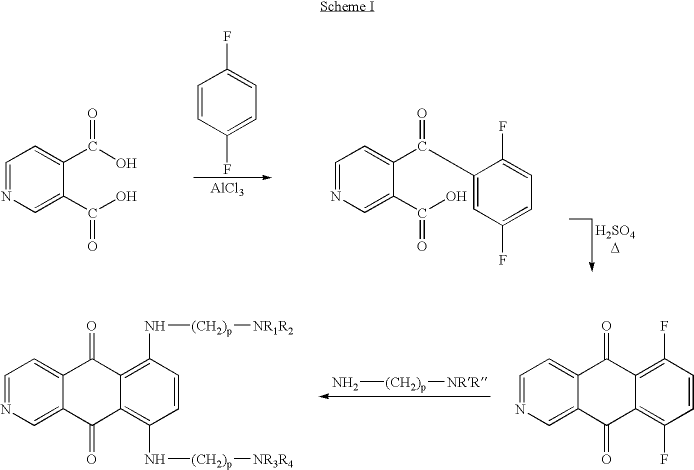

1,4-Difluoro-6-azanthracene-9,10-dione is prepared as generally presented above in Scheme I, and is reacted with a (N,N-disubstituted-amino)alkylamine in an appropriate solvent, such as pyridine or dry DMF, in a temperature range of 0° C. to reflux temperature (optionally under nitrogen atmosphere) for up to about 48 h to produce the 1,4-bis-{[(disubstituted-amino)alkyl]amino}-6-azaanthracene-9,10-dione product. The mixture is then poured into brine and stirred at room temperature for 30 min. The precipitate is collected by filtration, washed with 1 N NH₄OH, and dried under vacuum over KOH/silica for 15 h. This crude product is dissolved in CH₂Cl₂ and transferred to a silica gel flash column. Impurities such as the 6-azaanthracene-9,10-dione compounds that are substituted at only one of the 1- or 4-position, if present, are expected to elute before the desired product.

The desired product is eluted from the column and extracted successively with CH₂C₂/MeOH (10:1) and CH₂Cl₂/MeOH/Et₂N (90:10:1). The combined extracts are filtered and evaporated to give the desired 1,4-bis-{[(disubstituted-amino)alkyl]amino}-6-azaanthracene-9,10-dione.

The tertiary amine end product in Scheme I can be selectively oxidized using known oxidizing agents. Certain oxidizing agents that are known in the art for preparing the N-oxides from tertiary amine groups include, without limitation, potasium monopersulfate, monoperoxyphthalate acid, magnesium monoperoxyphthalate (MMPP), hydrogen peroxide, peracetic, trifluoroperacetic, perbenzoic, 3-chloroperbenzoic acid (CPBA), and 2-benzenesulfonyl-3-pheyloxaziridine (Davis reagent). The oxidation reaction can be carried out in a solvent such as chloroform, methylene chloride, 1,2-dichloroethane, or acetic acid, optionally in the presence of an alkali or alkaline-earth metal carbonate or bicarbonate. The reaction can be run from about 1 to 48 hours at a temperature of 0° C. to reflux temperature, and checked periodically for the presence of the desired bis-N-oxide. Depending on the groups bound to the amine, reaction times may need to be adjusted accordingly to obtain appropriate quantities of the desired bis-N-oxide product. See also Lee et al., “Nitracrine N-oxides: effects of variations in the nature of the side chain N-oxide on hypoxia-selective cytotoxicity” Anticancer Drug Des. 14 (6):487-497 (1999).

In one embodiment, the 1,4-bis-{[(disubstituted-amino)alkyl]amino}-6-azaanthracene-9,10-dione is stirred in CH₂Cl₂/MeOH (5:1) and is treated dropwise over about 30 min to about 2 h with a solution of 2-benzenesulfonyl-3-phenyloxaziridine (Davis reagent). After addition, the mixture is stirred at 20° C. in the dark for a further 90 min. It is then concentrated under reduced pressure at about 15-40° C. and then diluted successively with EtOAc and petroleum ether. The mixture is stirred at 20° C. for 15 min, then kept at −10° C. for 2 h. The precipitate is collected by filtration, washed with EtOAc/petroleum ether (1:1; 4 times), and suctioned dry. It is then dissolved in MeOH and the solution is treated with anhydrous HCl gas until it remains acidic (pH ca. 2). After storing at −10° C. overnight, the precipitate is collected by filtration and washed successively with MeOHJEtOAc (1:1; 5 times) and EtOAc (2 times), and dried under vacuum to give dihydrochloride product.

In addition to the dihydrochloride product, compounds having Formula IV can be provided as other pharmaceutically acceptable salts. Examples of pharmaceutically acceptable salts (i.e., addition salts) include inorganic and organic acid addition salts such as hydrochloride, hydrobromide, phosphate, sulphate, citrate, lactate, tartrate, maleate, fumarate, mandelate, benzoate and oxalate; and inorganic and organic base addition salts with bases such as sodium hydroxy, Tris(hydroxymethyl)aminomethane (TRIS, tromethane) and N-methyl-glucamine. Although the salts typically have similar physiological properties compared to the free base, certain acid addition salts may demonstrate preferred physicochemical properties, e.g., enhanced solubility, improved stability. One particular pharmaceutically acceptable salt is the maleate, such as the dimaleate.

Certain of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

In certain embodiments of the invention, compounds having Formula IV are administered in combination with one or more other active agents or treatments. By way of non-limiting example, a patient may be treated for a hyperproliferative disorder, such as cancer, by the administration of a therapeutically effective amount of a compound having Formula IV in combination with radiotherapy treatment or the administration of a second chemotherapeutic agent.

“In combination” refers to the use of more than one treatment. The use of the term “in combination” does not restrict the order in which treatments are administered to a subject being treated for a hyperproliferative disorder. A first treatment can be administered prior to, concurrently with, after, or within any cycling regimen involving the administration of a second treatment to a subject with a hyperproliferative disorder. For example, the first treatment can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before a treatment; or the first treatment can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after a second treatment. Such treatments include, for example, the administration of compounds having Formula IV in combination with one or more chemotherapeutic agents, radiotherapeutic agents and/or treatments, brachytherapy, radionuclide therapy and/or radiosurgery.

The term “chemotherapeutic agent,” as used herein, is intended to refer to any chemotherapeutic agent known to those of skill in the art to be effective for the treatment, prevention or amelioration of hyperproliferative disorders such as cancer. Chemotherapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. Any agent which is known to be useful, or which has been used or is currently being used for the treatment or amelioration of cancer can be used in combination with a compound having Formula IV. See, e.g., Hardman et al., eds., 1996, Goodman & Gilman's The Pharmacological Basis Of Therapeutics 9th Ed, Mc-Graw-Hill, New York, N.Y. for information regarding therapeutic agents which have been or are currently being used for the treatment or amelioration of cancer.

Particular chemotherapeutic agents useful in the methods and compositions of the invention include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, angiogenesis inhibitors and anti-angiogenic agents.

Certain chemotherapeutic agents include, but are not limited to, gemcitabine, pemetrexed, 5-fluorouracil, mitomycin C, doxorubicin, streptozocin, ifosfamide, cyclophosphamide, methotrexate, vincristine, and nitrosourea. Other chemotherapeutic agents that may be used include busulfan, temozolomide, chlorambucil, mechlorethane, polifeprosan, dactinomycin, epirubicin, idarubicin, valrubicin, plicamycin, cytarabine, floxuridine, thioguanine, mercaptopurine, cladribine, capecitabine, alemtuzumab, aldesleukin, ibritumomab, Avastin (bevacizumab), levamisole, dacarbazine, asparaginase, imatinib, trastuzumab, altretamine, procarbazine, gemtuzumab, mitoxantrone, pegaspargase, rituximab, interferon alpha-2a, methylprednisolone, alitretinoin, tretinoin, porfimer, arsenic trioxide, fludarabine, vinblastine, taxol, paclitaxel, docetaxel, melphalan, cisplatin, carboplatin, oxaliplatin, safriplatin, daunorubicin, etoposide, camptothecin, vinorelbine, topotecan, irinotecan, gifitinib, tarceva, and oblimersen.

Chemotherapeutic agents may be administered at doses that are recognized by those of skill in the art to be effective for the treatment of the cancer. In certain embodiments, chemotherapeutic agents may be administered at doses lower than those used in the art due to the additive or synergistic effect of the compounds having Formula IV. For example, gemcitabine can be administered at a dose of about 100, 200, 300, 400, 500, 600, 700, 800, 900, 1000, 1250, 1500, 1750, or 2000 mg/m2 by intravenous infusion over 30 minutes once weekly. A typical administration cycle for gemcitabine consists of infusions once weekly for three consecutive weeks followed by a week of rest from treatment.

The term “active vitamin D compound,” as used herein, is intended to refer to vitamin D which has been hydroxylated in at least the carbon-1 position of the A ring, e.g., 1α-hydroxyvitamin D₃. One particular active vitamin D compound for use in the present invention is 1α,25-dihydroxyvitamin D₃, also known as calcitriol. A large number of other active vitamin D compounds are known and can be used in the practice of the invention. Examples include 1α-hydroxy derivatives with a 17 side chain greater in length than the cholesterol or ergosterol side chains (see U.S. Pat. No. 4,717,721); cyclopentano-vitamin D analogs (see U.S. Pat. No. 4,851,401); vitamin D₃ analogues with alkynyl, alkenyl, and alkanyl side chains (see U.S. Pat. Nos. 4,866,048 and 5,145,846); trihydroxycalciferol (see U.S. Pat. No. 5,120,722); fluoro-cholecalciferol compounds (see U.S. Pat. No. 5,547,947); methyl substituted vitamin D (see U.S. Pat. No. 5,446,035); 23-oxa-derivatives (see U.S. Pat. No. 5,411,949); 19-nor-vitamin D compounds (see U.S. Pat. No. 5,237,110); and hydroxylated 24-homo-vitamin D derivatives (see U.S. Pat. No. 4,857,518). Particular examples include ROCALTROL (Roche Laboratories); CALCUEX injectable calcitriol; investigational drugs from Leo Pharmaceuticals including EB 1089 (24a,26a,27a-trihomo-22,24-diene-1α,25-(OH)₂-D₃, KH 1060 (20-epi-22-oxa-24a,26a,27a-trihomo-1α,25-(OH)₂-D₃), Seocalcitol, MC 1288 (1,25-(OH)₂-20-epi-D₃) and MC 903 (calcipotriol, 1α,24s-(OH)₂-22-ene-26,27-dehydro-D₃); Roche Pharmaceutical drugs that include 1,25-(OH)₂-16-ene-D₃, 1,25-(OH)₂-16-ene-23-yne-D₃, and 25-(OH)₂-16-ene-23-yne-D₃; Chugai Pharmaceuticals 22-oxacalcitriol (22-oxa-1α,25-(OH)₂-D₃; 1α-(OH)-D₅ from the University of Illinois; and drugs from the Institute of Medical Chemistry-Schering AG that include ZK 161422 (20-methyl-1,25-(OH)₂-D₃) and ZK 157202 (20-methyl-23-ene-1,25-(OH)₂-D₃); 1α-(OH)-D₂; 1α-(OH)-D₃ and 1α-(OH)-D₄. Additional examples include 1α,25-(OH)₂-26,27-d₆-D₃; 1α,25-(OH)₂-22-ene-D₃; 1α,25-(OH)₂-D₃; 1α,25-(OH)₂-D₂; 1α,25-(OH)₂-D₄; 1α,24,25-(OH)₃-D₃; 1α-24,25-(OH)₃-D₂; 1α,24,25-(OH)₃-D₄; 1α-(OH)-25-FD₃; 1α-(OH)-25-FD₄; 1α-(OH)-25-FD₂; 1α,24-(OH)₂-D₄; 1α,24-(OH)₂-D₃; 1α,24-(OH)₂-D₂; 1α,24-(OH)₂-25-FD₄; 1α,24-(OH)₂-25-FD₃; 1α,24-(OH)₂-25-FD₂; 1α,25-(OH)₂-26,27-F₆-22-ene-D₃; 1α, 25-(OH)₂-26,27-F₆-D₃; 1α, 25S—(OH)₂-26-F₃-D₃; 1α, 25-(OH)₂-24-F₂-D₃; 1α, 25S,26-(OH)₂-22-ene-D₃; 1α, 25R,26-(OH)₂-22-ene-D₃; 1α,25-(OH)₂-D₂; 1α, 25-(OH)₂-24-epi-D₃; 1α, 25-(OH)₂-23-yne-D₃; 1α, 25-(OH)₂-24R—F-D₃; 1α,25S,26-(OH)₂-D₃; 1α,24R—(OH)₂-25F-D₃; 1 (,25-(OH)₂-26,27-F₆-23-yne-D₃; 1α,25R—(OH)₂-26-F₃-D₃; 1α, 25,28-(OH)₃-D₂; 1α, 25-(OH)₂-16-ene-23-yne-D₃; 1α, 24R,25-(OH)₃-D₃; 1α, 25-(OH)₂-26,27-F₆-23-ene-D₃; 1α,25R—(OH)₂-22-ene-26-F₃-D₃; 1α, 25S—(OH)₂-22-ene-26-F₃-D₃; 1α, 25R—(OH)₂-D₃-26,26,26-d₃; 1α,25S—(OH)₂-D₃-26,26,26-d₃; and 1α,25R—(OH)₂-22-ene-D₃-26,26,26-d₃. Additional examples can be found in U.S. Pat. No. 6,521,608. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

The term “radiotherapeutic agent,” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy.

Brachytherapy can be administered according to any schedule, dose, or method known to one of skill in the art to be effective in the treatment or amelioration of cancer, without limitation. In general, brachytherapy comprises insertion of radioactive sources into the body of a subject to be treated for cancer, such as inside the tumor itself, such that the tumor is maximally exposed to the radioactive source, and minimizing the exposure of healthy tissue. Representative radioisotopes that can be administered in brachytherapy include, but are not limited to, phosphorus 32, cobalt 60, palladium 103, ruthenium 106, iodine 125, cesium 137, iridium 192, xenon 133, radium 226, californium 252, or gold 198. Methods of administering and apparatuses and compositions useful for brachytherapy are described in Mazeron et al., Sem. Rad. One. 12:95-108 (2002) and U.S. Pat. Nos. 6,319,189, 6,179,766, 6,168,777, 6,149,889, and 5,611,767.

Radionuclide therapy can be administered according to any schedule, dose, or method known to one of skill in the art to be effective in the treatment or amelioration of cancer, without limitation. In general, radionuclide therapy comprises systemic administration of a radioisotope that preferentially accumulates in or binds to the surface of cancerous cells. The preferential accumulation of the radionuclide can be mediated by a number of mechanisms, including, but not limited to, incorporation of the radionuclide into rapidly proliferating cells, specific accumulation of the radionuclide by the cancerous tissue without special targeting, or conjugation of the radionuclide to a biomolecule specific for a neoplasm.

Representative radioisotopes that can be administered in radionuclide therapy include, but are not limited to, phosphorus 32, yttrium 90, dysprosium 165, indium 111, strontium 89, samarium 153, rhenium 186, iodine 131, iodine 125, lutetium 177, and bismuth 213. While all of these radioisotopes may be linked to a biomolecule providing specificity of targeting, iodine 131, indium 111, phosphorus 32, samarium 153, and rhenium 186 may be administered systemically without such conjugation. One of skill in the art may select a specific biomolecule for use in targeting a particular neoplasm for radionuclide therapy based upon the cell-surface molecules present on that neoplasm. Examples of biomolecules providing specificity for particular cell are reviewed in an article by Thomas, Cancer Biother. Radiopharm. 17:71-82 (2002), which is incorporated herein by reference in its entirety. Furthermore, methods of administering and compositions useful for radionuclide therapy may be found in U.S. Pat. Nos. 6,426,400, 6,358,194, 5,766,571.

The term “radiotherapeutic treatment,” as used herein, is intended to refer to any radiotherapeutic treatment known to one of skill in the art to be effective to treat or ameliorate cancer, without limitation. For instance, the radiotherapeutic treatment can be external-beam radiation therapy, thermotherapy, radiosurgery, charged-particle radiotherapy, neutron radiotherapy, or photodynamic therapy.

External-beam radiation therapy can be administered according to any schedule, dose, or method known to one of skill in the art to be effective in the treatment or amelioration of cancer, without limitation. In general, external-beam radiation therapy comprises irradiating a defined volume within a subject with a high energy beam, thereby causing cell death within that volume. The irradiated volume preferably contains the entire cancer to be treated, and preferably contains as little healthy tissue as possible. Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652.

Thermotherapy can be administered according to any schedule, dose, or method known to one of skill in the art to be effective in the treatment or amelioration of cancer, without limitation. In certain embodiments, the thermotherapy can be cryoablation therapy. In other embodiments, the thermotherapy can be hyperthermic therapy. In still other embodiments, the thermotherapy can be a therapy that elevates the temperature of the tumor higher than in hyperthermic therapy.

Cryoablation therapy involves freezing of a neoplastic mass, leading to deposition of intra- and extra-cellular ice crystals; disruption of cellular membranes, proteins, and organelles; and induction of a hyperosmotic environment, thereby causing cell death. Methods for and apparatuses useful in cryoablation therapy are described in Murphy et al., Sem. Urol. Oncol. 19:133-140 (2001) and U.S. Pat. Nos. 6,383,181, 6,383,180, 5,993,444, 5,654,279, 5,437,673, and 5,147,355.

Hyperthermic therapy typically involves elevating the temperature of a neoplastic mass to a range from about 42° C. to about 44° C. The temperature of the cancer may be further elevated above this range; however, such temperatures can increase injury to surrounding healthy tissue while not causing increased cell death within the tumor to be treated. The tumor may be heated in hyperthermic therapy by any means known to one of skill in the art without limitation. For example, and not by way of limitation, the tumor may be heated by microwaves, high intensity focused ultrasound, ferromagnetic thermoseeds, localized current fields, infrared radiation, wet or dry radiofrequency ablation, laser photocoagulation, laser interstitial thermic therapy, and electrocautery. Microwaves and radiowaves can be generated by waveguide applicators, horn, spiral, current sheet, and compact applicators.

Other methods, apparatuses and compositions for raising the temperature of a tumor are reviewed in an article by Wust et al., Lancet Oncol. 3:487-97 (2002), and described in U.S. Pat. Nos. 6,470,217, 6,379,347, 6,165,440, 6,163,726, 6,099,554, 6,009,351, 5,776,175, 5,707,401, 5,658,234, 5,620,479, 5,549,639, and 5,523,058.

Radiosurgery can be administered according to any schedule, dose, or method known to one of skill in the art to be effective in the treatment or amelioration of cancer, without limitation. In general, radiosurgery comprises exposing a defined volume within a subject to a manually directed radioactive source, thereby causing cell death within that volume. The irradiated volume preferably contains the entire cancer to be treated, and preferably contains as little healthy tissue as possible. Typically, the tissue to be treated is first exposed using conventional surgical techniques, then the radioactive source is manually directed to that area by a surgeon. Alternatively, the radioactive source can be placed near the tissue to be irradiated using, for example, a laparoscope. Methods and apparatuses useful for radiosurgery are further described in Valentini et al., Eur. J. Surg. Oncol. 28:180-185 (2002) and in U.S. Pat. Nos. 6,421,416, 6,248,056, and 5,547,454.

Charged-particle radiotherapy can be administered according to any schedule, dose, or method known to one of skill in the art to be effective in the treatment or amelioration of cancer, without limitation. In certain embodiments, the charged-particle radiotherapy can be proton beam radiotherapy. In other embodiments, the charged-particle radiotherapy can be helium ion radiotherapy. In general, charged-particle radiotherapy comprises irradiating a defined volume within a subject with a charged-particle beam, thereby causing cellular death within that volume. The irradiated volume preferably contains the entire cancer to be treated, and preferably contains as little healthy tissue as possible. A method for administering charged-particle radiotherapy is described in U.S. Pat. No. 5,668,371.

Neutron radiotherapy can be administered according to any schedule, dose, or method known to one of skill in the art to be effective in the treatment or amelioration of cancer, without limitation. In certain embodiments, the neutron radiotherapy can be a neutron capture therapy. In such embodiments, a compound that emits radiation when bombarded with neutrons and preferentially accumulates in a neoplastic mass is administered to a subject. Subsequently, the tumor is irradiated with a low energy neutron beam, activating the compound and causing it to emit decay products that kill the cancerous cells. The compound to be activated can be caused to preferentially accumulate in the target tissue according to any of the methods useful for targeting of radionuclides, as described above, or in the methods described in Laramore, Semin. Oncol. 24:672-685 (1997) and in U.S. Pat. Nos. 6,400,796, 5,877,165, 5,872,107, and 5,653,957.

In other embodiments, the neutron radiotherapy can be a fast neutron radiotherapy. In general, fast neutron radiotherapy comprises irradiating a defined volume within a subject with a neutron beam, thereby causing cellular death within that volume.

Photodynamic therapy can be administered according to any schedule, dose, or method known to one of skill in the art to be effective in the treatment or amelioration of cancer, without limitation. In general, photodynamic therapy comprises administering a photosensitizing agent that preferentially accumulates in a neoplastic mass and sensitizes the neoplasm to light, then exposing the tumor to light of an appropriate wavelength. Upon such exposure, the photosensitizing agent catalyzes the production of a cytotoxic agent, such as, e.g., singlet oxygen, which kills the cancerous cells. Methods of administering and apparatuses and compositions useful for photodynamic therapy are disclosed in Hopper, Lancet Oncol. 1:212-219 (2000) and U.S. Pat. Nos. 6,283,957, 6,071,908, 6,011,563, 5,855,595, 5,716,595, and 5,707,401.

Radiotherapy can be administered to destroy tumor cells before or after surgery, before or after chemotherapy, and sometimes during chemotherapy. Radiotherapy may also be administered for palliative reasons to relieve symptoms of cancer, for example, to lessen pain. Among the types of tumors that can be treated using radiotherapy are localized tumors that cannot be excised completely and metastases and tumors whose complete excision would cause unacceptable functional or cosmetic defects or be associated with unacceptable surgical risks.

It will be appreciated that both the particular radiation dose to be utilized in treating cancer and the method of administration will depend on a variety of factors. Thus, the dosages of radiation that can be used according to the methods of the present invention are determined by the particular requirements of each situation. The dosage will depend on such factors as the size of the tumor, the location of the tumor, the age and sex of the patient, the frequency of the dosage, the presence of other tumors, possible metastases and the like. Those skilled in the art of radiotherapy can readily ascertain the dosage and the method of administration for any particular tumor by reference to Hall, E. J., Radiobiology for the Radiologist, 5th edition, Lippincott Williams & Wilkins Publishers, Philadelphia, Pa., 2000; Gunderson, L. L. and Tepper J. E., eds., Clinical Radiation Oncology, Churchill Livingstone, London, England, 2000; and Grosch, D. S., Biological Effects of Radiation, 2nd edition, Academic Press, San Francisco, Calif., 1980. In certain embodiments, radiotherapeutic agents and treatments may be administered at doses lower than those known in the art due to the additive or synergistic effect of the active vitamin D compound.

Compositions in accordance with the present invention may be employed for administration in any appropriate manner, e.g., oral or buccal administration, e.g., in unit dosage form, for example in the form of a tablet, in a solution, in hard or soft encapsulated form including gelatin encapsulated form, sachet, or lozenge. Compositions may also be administered parenterally or topically, e.g., for application to the skin, for example in the form of a cream, paste, lotion, gel, ointment, poultice, cataplasm, plaster, dermal patch or the like, or for ophthalmic application, for example in the form of an eye-drop, -lotion or -gel formulation. Readily flowable forms, for example solutions, emulsions and suspensions, may also be employed e.g., for intralesional injection, or may be administered rectally, e.g., as an enema or suppository, or intranasal administration, e.g., as a nasal spray or aerosol. Microcrystalline powders may be formulated for inhalation, e.g., delivery to the nose, sinus, throat or lungs. Transdermal compositions/devices and pessaries may also be employed for delivery of the compounds of the invention. The compositions may additionally contain agents that enhance the delivery of the compounds having Formula IV (or other active agents), e.g., liposomes, polymers or co-polymers (e.g., branched chain polymers).

The pharmaceutical compositions of the present invention may further comprise one or more additives. Additives that are well known in the art include, e.g., detackifiers, anti-foaming agents, buffering agents, antioxidants (e.g., ascorbic acid, ascorbyl palmitate, sodium ascorbate, butylated hydroxyanisole (BHA), butylated hydroxytoluene (BHT), propyl gallate, malic acid, fumaric acid, potassium metabisulfite, sodium bisulfite, sodium metabisulfite, and tocopherols, e.g., α-tocopherol (vitamin E)), preservatives, chelating agents, viscomodulators, tonicifiers, flavorants, colorants, odorants, opacifiers, suspending agents, binders, fillers, plasticizers, lubricants, and mixtures thereof. The amounts of such additives can be readily determined by one skilled in the art, according to the particular properties desired, and can be formulated such that compounds having Formula IV are stable, e.g., not reduced by antioxidant additives.

The additive may also comprise a thickening agent. Suitable thickening agents may be of those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents may not be required. Use of thickening agents is, on the other hand, indicated, e.g., where topical application is foreseen.

When an active vitamin D compound is used in the practice of the invention, a pharmaceutical composition is provided comprising (a) a lipophilic phase component, (b) one or more surfactants, (c) an active vitamin D compound; wherein said composition is an emulsion pre-concentrate, which upon dilution with water, in a water to composition ratio of about 1:1 or more of said water, forms an emulsion having an absorbance of greater than 0.3 at 400 nm. The pharmaceutical composition of the invention may further comprise a hydrophilic phase component. Such pharmaceutical compositions are described in PCT International Application Publication No. WO 03/047595.

In certain aspects of the invention, pharmaceutical compositions of the invention comprise a compound having Formula I, an active vitamin D compound, a lipophilic component, and a surfactant. The lipophilic component may be present in any percentage from about 1% to about 100%. The lipophilic component may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant may be present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In other embodiments, the pharmaceutical compositions comprise 50% MIGLYOL 812 and 50% vitamin E TPGS, 90% MIGLYOL 812 and 10% vitamin E TPGS, or 95% MIGLYOL 812 and 5% vitamin E TPGS.

In another embodiment of the invention, the pharmaceutical compositions comprise a compound having Formula 1V, an active vitamin D compound and a lipophilic component, e.g., around 100% MIGLYOL 812.

Although the dosage of the compound having Formula IV will vary according to the activity and/or toxicity of the particular compound, the condition being treated, and the physical form of the pharmaceutical composition being employed for administration, it may be stated by way of guidance that a dosage selected in the range from 0.1 to 20 mg/kg of body weight per day will often be suitable, although higher dosages, such as 0.1 to 50 mg/kg of body weight per day may be useful. Those of ordinary skill in the art are familiar with methods for determining the appropriate dosage. Methods for assessing the toxicity, activity and/or selectivity of the compounds having Formula IV may be carried out as described in Lee et al., supra, and PCT Published International Application WO 92/15300, supra, and may be useful for approximating and/or determining dose ranges for compounds having Formula IV.

In certain instances, the dosage of the compounds having Formula IV may be lower, e.g., when used in combination with at least a second hyperproliferative disorder treatment, and may vary according to the activity and/or toxicity of the particular compound, the condition being treated, and the physical form of the pharmaceutical composition being employed for administration.

When the unit dosage form of the composition is a capsule, the total quantity of ingredients present in the capsule is some instances about 10-1000 μL. In other instances, the total quantity of ingredients present in the capsule is about 100-300 μL.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative individually inventive teachings only and not as not limiting the invention in its broadest aspect.

The amount of active vitamin D compound in compositions of the invention will of course vary, e.g., depending on the intended route of administration and to what extent other components are present. In general, however, the active vitamin D compound will suitably be present in an amount of from about 0.005% to 20% by weight based upon the total weight of the composition. In certain embodiments, the active vitamin D compound is present in an amount of from about 0.01% to 15% by weight based upon the total weight of the composition.

The pharmaceutical compositions of the invention may be in a liquid formulation. Liquid formulations within the scope of the invention may comprise, e.g., a lipophilic phase component present in an amount of from about 50% to about 60% by weight based upon the total weight of the composition, a surfactant present in an amount of from about 4% to about 25% by weight based upon the total weight of the composition, a compound of Formula IV as described above, optionally with an active vitamin D compound present in an amount of from about 0.01% to about 15% by weight based upon the total weight of the composition, and a hydrophilic phase component present in an amount of from about 5% to about 10% by weight based upon the total weight of the composition.

In addition to the foregoing the present invention also provides a process for the production of a pharmaceutical composition as hereinbefore defined, which process comprises bringing the individual components thereof into intimate admixture and, when required, compounding the obtained composition in unit dosage form, for example filling said composition into tablets, gelatin, e.g., soft or hard gelatin, capsules, or non-gelatin capsules.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1. A compound having Formula IV: 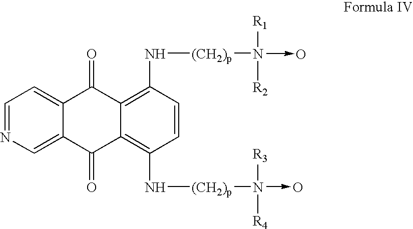
or a pharmaceutically acceptable salt thereof, wherein:
R₁, R₂, R₃ and R₄ are independently C₁₋₄ alkyl, C₂₋₄ hydroxyalkyl, C₂₋₄ dihydroxyalkyl in which the carbon atom attached to the nitrogen atom does not carry a hydroxy group and no carbon atom is substituted by two hydroxy groups,
or R₁ and R₂ can be taken together as a C₂₋₆ alkylene or C₄₋₆ alkylidene with the nitrogen atom to which R₁ and R₂ are attached to form a heterocycle having 3 to 7 atoms in the ring;
and/or R₃ and R₄ can be taken together as a C₂₋₆ alkylene or C₄₋₆ alkylidene with the nitrogen atom to which R₃ and R₄ are attached to form a heterocycle having 3 to 7 atoms in the ring; and
p is 2 to 4.

2. The compound of claim 1, wherein R₁-R₄ are C₁₋₄ alkyl.

3. The compound of claim 2, wherein C₁₋₄ alkyl is selected from the group consisting of methyl, ethyl, propyl, isopropyl, and butyl.

4. The compound of claim 1, wherein the compound is selected from the group consisting of:
1,4-bis-{[2-(dimethylamino-N-oxide)ethyl]amino}-6-azaanthracene-9,10-dione;
1,4-bis-{[2-(diethylamino-N-oxide)ethyl]amino}-6-azaanthracene-9,10-dione;
1,4-bis-{[2-(dipropylamino-N-oxide)ethyl]amino}-6-azaanthracene-9,10-dione;
1,4-bis-{[2-(diisopropylamino-N-oxide)ethyl]amino}-6-azaanthracene-9,10-dione;
1,4-bis-{[2-(dibutylamino-N-oxide)ethyl]amino}-6-azaanthracene-9,10-dione;
1,4-bis-{[2-(dipiperidin-1-yl-N-oxide)ethyl]amino}-6-azaanthracene-9,10-dione;
1,4-bis-{[2-(dimorpholin-4-yl-N-oxide)ethyl]amino}-6-azaanthracene-9,10-dione;
1,4-bis-{[2-(aziridin-1-yl-N-oxide)ethyl]amino}-6-azaanthracene-9,10-dione;
1,4-bis-{[3-(dimethylamino-N-oxide)propyl]amino}-6-azaanthracene-9,10-dione;
1,4-bis-{[3-(diethylamino-N-oxide)propyl]amino}-6-azaanthracene-9,10-dione;
1,4-bis-{[3-(dipropylamino-N-oxide)propyl]amino}-6-azaanthracene-9,10-dione;
1,4-bis-{[3-(diisopropylamino-N-oxide)propyl]amino}-6-azaanthracene-9,10-dione;
1,4-bis-{[3-(dibutylamino-N-oxide)propyl]amino}-6-azaanthracene-9,10-dione;
1,4-bis-{[3-(dipiperidin-1-yl-N-oxide)propyl]amino}-6-azaanthracene-9,10-dione;
1,4-bis-{[3-(dimorpholin-4-yl-N-oxide)propyl]amino}-6-azaanthracene-9,10-dione; and
1,4-bis-{[3-(aziridin-1-yl-N-oxide)propyl]amino}-6-azaanthracene-9,10-dione.

5. A pharmaceutical composition comprising the compound of claim 1.

6. The pharmaceutical composition of claim 5, further comprising one or more other active agents.

7. The pharmaceutical composition of claim 6, wherein the one or more other active agents is an active vitamin D compound.

8. The pharmaceutical composition of claim 6, wherein the one or more other active agents is a chemotherapeutic agent.

9. The pharmaceutical composition of claim 8, wherein the second chemotherapeutic agent is selected from the group consisting of gemcitabine, pemetrexed, 5-fluorouracil, mitomycin C, doxorubicin, streptozocin, ifosfamide, cyclophosphamide, methotrexate, vincristine, nitrosourea, busulfan, temozolomide, chlorambucil, mechlorethane, polifeprosan, dactinomycin, epirubicin, idarubicin, valrubicin, plicamycin, cytarabine, floxuridine, thioguanine, mercaptopurine, cladribine, capecitabine, alemtuzumab, aldesleukin, ibritumomab, Avastin (bevacizumab), levamisole, dacarbazine, asparaginase, imatinib, trastuzumab, altretamine, procarbazine, gemtuzumab, mitoxantrone, pegaspargase, rituximab, interferon alpha-2a, methylprednisolone, alitretinoin, tretinoin, porfimer, arsenic trioxide, fludarabine, vinblastine, taxol, paclitaxel, docetaxel, melphalan, cisplatin, carboplatin, oxaliplatin, daunorubicin, etoposide, camptothecin, vinorelbine, topotecan, irinotecan, gifitinib, tarceva, and oblimersen.

10. A method of treating, preventing or ameliorating a hyperproliferative disorder comprising administering to a animal in need thereof a therapeutically effective amount of the compound of claim 1.

11. The method of claim 10, wherein the hyperproliferative disorder is cancer.

12. The method of claim 11, wherein the cancer is a carcinoma, leukemia, lymphoma, melanoma or sarcoma.

13. The method of claim 11, wherein the cancer is of the bladder, brain, breast, cervix, colon, endometrium, esophagus, head and neck, kidney, larynx, liver, lung, oral cavity, ovaries, pancreas, prostate, skin, stomach, or testis.

14. The method of claim 11, wherein the cancer is selected from the group consisting of acute and chronic lymphocytic leukemias, acute granulocytic leukemia, adrenal cortex carcinomas, bladder carcinomas, breast carcinomas, cervical carcinomas, cervical hyperplasia, choriocarcinomas, chronic granulocytic leukemia, chronic lymphocytic leukemia, colon carcinomas, endometrial carcinomas, esophageal carcinomas, essential thrombocytosis, genitourinary carcinomas, hairy cell leukemia, head and neck carcinomas Hodgkin's disease, Kaposi's sarcoma, lung carcinomas, lymphoma, malignant carcinoid carcinomas, malignant hypercalcemia, malignant melanomas, malignant pancreatic insulinoma, medullary thyroid carcinoma, melanoma, multiple myeloma, mycosis fingoides, myeloid and lymphocytic leukemia, neuroblastoma, non-Hodgkin's lymphomas, osteogenic sarcoma, ovarian carcinomas, pancreatic carcinomas, polycythemia vera, primary brain carcinomas, primary macroglobulinemia, prostatic carcinomas, renal cell carcinomas, rhabdomyosarcoma, skin cancer, small-cell lung carcinomas, soft-tissue sarcomas, squamous cell carcinoma, stomach carcinomas, testicular carcinomas, thyroid carcinomas, and Wilms' tumor.

15. The method of claim 11, further comprising administering one or more other active agents or treatments to the animal.

16. The method of claim 15, wherein the one or more other active agents or treatments comprises administering an active vitamin D compound.

17. The method of claim 15, wherein the one or more other active agents or treatments are independently selected from the group consisting of a chemotherapeutic agent, radiotherapeutic agent, brachytherapy, radionuclide therapy and radiosurgery.

18. The method of claim 10, wherein the hyperproliferative disorder is age-related macular degeneration, Crohn's disease, cirrhosis, a chronic inflammatory-related disorder, diabetic retinopathy, granulomatosis, inflammatory bowel disease, psoriasis, rheumatoid arthritis, systemic lupus erythematosus (SLE), vascular hyperproliferation secondary to retinal hypoxia, or vasculitis.

19. The method of claim 10, wherein the hyperproliferative disorder is an immunoproliferative disorder.

20. The method of claim 10, wherein the hyperproliferative disorder is immune hyperproliferation associated with organ or tissue transplantation.
